(19) United States
(12) Patent Application Publication
(10) Pub. No.: US 2022/0066190 A1
(43) Pub. Date: Mar. 3, 2022

(54) SLIDES FOR CALIBRATION OF MFISH

(71) Applicant: Applied Materials, Inc., Santa Clara, CA (US)

(72) Inventors: Chloe Kim, San Jose, CA (US); Lu Yan, Cupertino, CA (US)

(21) Appl. No.: 17/461,827

(22) Filed: Aug. 30, 2021

Related U.S. Application Data
(60) Provisional application No. 63/072,908, filed on Aug. 31, 2020.

(51) Int. Cl. G02B 21/34; G01N 21/64

(52) U.S. Cl. CPC G02B 21/34; C12Q 1/6841; G01N 2021/6482; G01N 21/6458

(57) ABSTRACT

A calibration slide for calibrating a multiplexed fluorescence in-situ hybridization (FISH) system has a substrate having a surface and a first plurality of beads on the surface. Each bead of the first plurality of beads has at least one binding domain to be bound to a corresponding probe that has a tag and a targeting domain that binds to a binding domain.

### CROSS-REFERENCE TO RELATED APPLICATIONS

This application claims priority to U.S. Provisional Application Ser. No. 63/072,908, filed on Aug. 31, 2020, the disclosure of which is incorporated by reference.

### TECHNICAL FIELD

This disclosure relates to calibrating a multiplexed fluorescence in-situ hybridization (FISH) system by using calibration slides to calibrate the system without the sample, prior to determining the intensity and/or spatial location of analytes within a sample.

### BACKGROUND

Fluorescence in situ hybridization (FISH) assays are a molecular cytogenetic method used in the detection and quantification of the presence or absence of specific nucleic acid (DNA or RNA) sequences in a sample, commonly used as a diagnostic tool in medicine and research. For example, FISH is used to detect the presence of chromosomal aberrations (gene mutations), changes in the expression profile and location of genes associated with different disease states (e.g. cancer, autoimmune disorders, psychiatric disorders, etc.), and species identification.

FISH involves the use of fluorescence microscopy, where the sample of interest is labeled with nucleic acid probes with high complementarity to bind to the genetic sequence of interest. These probes, or separate readout probes that bind to the nucleic acid probes, are labeled with a fluorophore, that when excited by an excitation source (such as from a fluorescent microscopy apparatus), emits a fluorescent signal. The signal is then detected and processed to convert the signals into an optical image, which typically depicts the spatial location and/or abundance of the analyte in the sample.

In particular, as part of FISH imaging, a sample is exposed to multiple oligonucleotide probes. During a round of hybridization, different oligonucleotide probes target different nucleotide sequences. Then a round of fluorescence images can be acquired by sequentially exposing the sample to excitation light of different wavelengths to excite the different probes. In addition, after a round of images is obtained, the probes can be photobleached, and another round of hybridization can be performed using more oligonucleotide probes that target still different nucleotide sequences, followed by another round of imaging.

For each given pixel, its fluorescence intensities from the different images form a signal sequence. This sequence is then compared to a library of reference codes from a codebook that associates each code with a gene. The best matching reference code can be used to identify an associated gene that is expressed at that pixel in the image.

### SUMMARY

In one aspect, a method of calibrating a fluorescence in-situ hybridization (FISH) system includes a) contacting a calibration slide with a plurality of probes, wherein each probe comprises a tag and a targeting domain, wherein the targeting domain binds a binding domain, wherein the calibration slide comprises a surface comprising a first plurality of beads, and wherein each bead has (i) a plurality of binding domains or (ii) no binding domains, b) obtaining one or more images of the calibration slide, and c) calibrating the FISH system based on the one or more images.

In another aspect, a method of calibrating a fluorescence in-situ hybridization (FISH) system includes contacting a calibration slide with a plurality of probes, wherein the calibration slide comprises a surface having a first plurality of beads, wherein each bead of the first plurality of beads has one or more binding domains, wherein each probe has a tag and a targeting domain, and wherein the targeting domain binds a binding domain from the at least one binding domain, (b) obtaining one or more images of the calibration slide, and (c) calibrating the FISH system based on the one or more images.

In another aspect, a calibration slide for calibrating a multiplexed FISH system includes a surface and a plurality of beads. Each bead has (i) a plurality of binding domains or (ii) no binding domains, and each binding domain binds to one or more probes, wherein each probe comprises a tag and a targeting domain, and each targeting domain binds to a binding domain, if present.

Implementations may include one or more of the following features.

Each bead of the first plurality of beads may have exactly one binding domain. Each bead of the first plurality of beads may have a first binding domain and a second binding domain. The surface of the calibration slide has a second plurality of beads that have no binding domains.

The plurality of probes may include a plurality of first probes, wherein each first probe comprises a first tag and a first targeting domain, wherein the first targeting domain binds a first binding domain. A second probe may have a second targeting domain specifically binds a second bead comprising a second binding domain. The plurality of probes may include a plurality of first probes and plurality of second probes. Each first probe may have a first tag and a first targeting domain that binds the first binding domain, and each second probe may have a second tag and a second targeting domain that binds a second binding domain. The plurality of first probes and the plurality of second probes may be present in different concentrations.

After obtaining one or more images and before calibrating the FISH system, the calibration slide may be contacted with a plurality of second probes. Each second probe may have a second tag and a second targeting domain that binds the second binding domain. A second image of the calibration slide may be obtained. The calibration slide may be contacted with a mixture comprising first probes and second probes.

After obtaining the one or more images and before calibrating the FISH system, the calibration slide may be washed. The washing may be after obtaining the second image. ing step after step (b) and before step (c). Before calibrating the FISH system, step (a) may be repeated to obtain a third image of the calibration slide.

Each binding domain may independently include an oligonucleotide. Each targeting domain may independently include an oligonucleotide. Each oligonucleotide may independently be DNA or RNA. Each oligonucleotide may independently be DNA. Each oligonucleotide may independently be RNA. Each oligonucleotide may independently have 15-30 residues. The oligonucleotide sequence of each first targeting domain may be complementary to the oligonucleotide sequence of each first binding domain. The oligonucleotide sequence of each second targeting domain may be complementary to the oligonucleotide sequence of each second binding domain.

The plurality of beads may include comprise one or more first beads and one or more second beads. Each first bead and each second bead may have a different number of binding domains. The one or more first beads and the one or more second beads may be secured to specific locations of the calibration slide.

Each bead may have a carboxyl latex core. The carboxyl latex core may have a diameter of between 0.1 μm and 1 μm.

Calibrating the FISH system may include determining the binding efficiency between a probe and a binding domain. Calibrating the FISH system may include determining appropriate image acquisition conditions. Calibrating the FISH system may include determining multi-color brightness and photostability of one or more fluorescent moieties. Calibrating the FISH system may include determining the conjugation efficiency between a first fluorescent moiety and a first probe. Calibrating the FISH system may include determining the optimal number of binding domains on a bead in order to reach a balanced signal. Calibrating the FISH system may include generating template images to test spot calling algorithm developed for a quantitative analysis.

Advantages may include, but are not limited to, one or more of the following. The calibration slide can be used to calibrate and validate a multiplexed fluorescence in-situ hybridization assay and the imaging system in a reproducible manner without using samples. The system can be calibrated across a variety of parameters, such as oligo-to-oligo binding efficiency, dye-to-probe conjugation efficiency, on-stage hybridization, fluorescence intensities, photostability, focal calibration, and/or optimization of image acquisition configurations. The calibration slides described herein can avoid the variabilities and uncertainties associated with calibrating a FISH system based on biological samples.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 depicts a diagram showing different components of an automated microfluidics and image acquisition system.

FIG. 2 are representative designs of oligonucleotide-nanoparticle calibration slides.

FIG. 3 illustrates a representative set of fluorescent microscopy images of oligonucleotide-nanoparticles acquired sequentially and overlaid. Six cycles were performed, each cycle comprising (a) hybridizing one or more single-color probes (e.g., an oligonucleotide covalently linked to a unique fluorescent dye; and (b) imaging one or more single-color probes.

FIG. 4 depicts a multiplexed fluorescent in-situ hybridization (mFISH) imaging and image processing apparatus.

### DETAILED DESCRIPTION

Current methods of fluorescence microscope calibration require extensive optimization to ensure that the components used in the assay bind the desired target with sufficient specificity, but do not detrimentally interact with each other. This is particularly important in a clinical setting, where such detrimental interactions during a calibration procedure could, for example, provide a false positive or false negative readout on the assay. Given the clinical importance of these screening methods, there is an unmet need for selecting appropriate assay components, for example, through an improved calibration process.

The present application addresses this need by providing compositions and methods that can be used to calibrate a multiplexed fluorescence in-situ hybridization system in vitro without using the sample, thus providing improved assessment of oligo-to-oligo binding efficiency and dye-to-probe conjugation efficiency, permitting on-stage hybridization, improved assessment of fluorescence intensities and photo stability, and increased focal calibration and optimization of image acquisition configurations.

In particular, a calibration slide can be used to calibrate a multiplexed fluorescence in-situ hybridization system, which in turn, is used to determine the number and spatial location of analytes within a sample. The calibration slides are designed for calibrating both a multiplexed fluorescence in-situ hybridization assay and the imaging instrument, without the need to use a sample.

The calibration slide includes a substrate and a surface supporting a plurality of beads. Each bead can have one or more binding domains, and each binding domain binds to a probe. Each probe can include a fluorophore and a targeting domain, where the targeting domain binds to a binding domain. The binding domain and corresponding targeting domain can each be a oligonucleotide sequence.

### Overview

Hypothetically, the intensity of light emitted from a sample treated with probes and exposed to excitation light would be proportional to the expression of the targeted nucleotide sequence in the sample. Thus, the intensity of the light in various pixels of a FISH image could be used determine the degree of expression of the targeted nucleotide sequence at that pixel. This information can be used, in turn, to spatially determine expression of genes within a sample.

However, the values of various parameters that can affect the intensity of the light emitted during the FISH imaging may be unknown or can vary from system to system. For example, the oligo-to-oligo binding efficiency of a targeting probe to the sample, or the oligo-to-oligo binding efficiency of the readout probe to the sample or the targeting probe, may initially be unknown or can vary depending on environmental conditions, e.g., the exact chemistry, temperature, etc. Moreover, the image acquisition conditions can vary from system to system. For example, off-the-shelf excitation light sources that are ostensibly supposed emit at the same wavelength and intensity could, in fact, have manufacturing differences and be subject to drift, leading to differences in excitation conditions for fluorophores and thus different emitted intensities despite ostensibly identical optical systems.

Some techniques to ameliorate these issues include microscope calibration slides that are pre-coated with fluorescent film or fluorescent particles. However, these approaches still require a sample as a control, which leads to sample-to-sample variability in the image and other uncertainties resulting from sample preparation, size, and other factors. Thus, improved techniques are needed for calibrating of fluorescence microscopy systems.

An approach that may address some of these issues is to use a calibration slide to which a plurality of beads are secured, with each bead having a plurality of binding domains configured to specifically bind to the targeting domain of at least one probe to be used in the FISH imaging process. The purpose of this calibration slide is to develop ‘synthetic’ testing sample (controlled system) and avoid variations from biological samples.

When the calibration slide is exposed to the probes and the probes are activated by excitation light, the emitted light intensity can be measured. Because the beads can have binding domains at a known density, the measure of light intensity from the probes provides a calibration measurement that is particular to the operating conditions. For example, knowing the light intensity value given the binding domain density of the bead permits calculation of the density of the targeted nucleotide in the sample from the measured light intensity of the sample.

### Definitions

Unless otherwise specified, all numbers expressing quantities of ingredients, reaction conditions, and other properties or parameters used in the specification and claims are to be understood as being modified in all instances by the term “about.” Accordingly, unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques. The term “about” encompasses variations of ±10% of the numerical value of the number.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

A “fluorescent moiety” refers to a substance that emits light of a particular wavelength (e.g., having a particular color) in response to being exposed to a light of a wavelength that “excites” the fluorescent moiety. A fluorescent moiety can be coupled to other molecules (e.g., DNA molecules, proteins, antibodies, or beads). A fluorescence microscope is an optical microscope that generates an image by exposing a fluorescent moiety to light of a wavelength that excites the fluorescent moiety. Exemplary fluorescent moieties include pacific blue (PacB), Horizon V450, pacific orange (PacO), aminomethylcoumarin acetate (AMCA), fluorescein isothiocyanate (FITC), Alexa488, phycoerythrin (PE), peridinin chlorophyl protein/cyanine 5.5 (PerCP-Cy5.5), PerCP, PE-TexasRed, phycoerythrin/cyanine7 (PE-Cy7), allophycocyanine (APC), Alexa594, cyanine 5.5 (Cy5.5), IR800, Alexa647, allophycocyanine/H7 (APC-H7), APC-Cy7, Alexa680 and Alexa700.

A “binding domain” on a bead refers to a structure on the bead that can bind to a probe, as described herein. Exemplary binding domains include, but are not limited to, oligonucleotides such as DNA and RNA. In some embodiments, “bind” or “binding” refers to the interaction of oligonucleotides having sufficient complementarity to hybridize.

A “tag” on a probe refers to a handle for detecting the probe, for example, a fluorescent tag or an affinity tag. In some embodiments, the tag comprises a fluorescent moiety. In some embodiments, the tag comprises an affinity tag. In some embodiments, the affinity tag is biotin. When biotin is the affinity tag, the probe may be pulled down using beads coated with streptavidin, or detected by the addition of streptavidin labeled with a fluorescent moiety.

“Calibrating” the multiplexed FISH system includes the assessment various parameters, which will vary according to the particular system and configuration. These parameters include, but are not limited to, oligo-to-oligo binding efficiency, dye-to-probe conjugation efficiency, control of fluidics, on-stage flowcell hybridization, fluorescence intensities, photostability, focal calibration, and/or optimization of image acquisition configurations. In some embodiments, calibrating the multiplexed FISH system involves adjusting the parameters of an image processing algorithm. In some embodiments, the calibration is based on at least one image of the calibration slide and predetermined data concerning the plurality of beads. For example, such a predetermined data concerning the plurality of beads includes the size of the beads, the bead-to-oligonucleotide ratio during bead preparation, and where certain types of beads are located on the calibration slide. In some embodiments, an image of the calibration slide and the predetermined data are used to set one or more parameters of an image processing algorithm (e.g., the spatial intensity distribution of the fluorescent region).

“Binding efficiency” refers to the binding affinity between two molecules. In some embodiments, the binding efficiency between a probe and a bead can be determined by contacting a calibration slide having a plurality of the beads to a plurality of the probes and measuring the amount of fluorescence signal after a certain washing step.

“Image acquisition conditions” include, in a non-limiting way, the intensity and wavelengths of excitation lights, the selection of imaging buffer, the selection of the microscope, exposure time, grayscale levels, image frame resolution, optical zoom and the selection of color filters. For live cell imaging, the appropriate image acquisition conditions also include appropriate environmental conditions such as temperature, media, CO₂ and possibly perfusion.

“Photostability” refers to the stability of the signal produced by a fluorescent moiety. Photobleaching is the irreversible destruction of a fluorophore under the influence of light. Any fluorescent molecule will photobleach at some point. In addition to photobleaching, a fluorescent moiety may display reversible intensity changes and photoswitching, which usually are undesirable properties. Ideally, a fluorescent moiety should emit a stable fluorescent signal, showing little or no deterioration or change of the signal during the course of an experiment. The photostability of a fluorescent moiety can be measured by measuring fluorescence intensity over time using time-lapse imaging. For cell imaging, it is desirable to have fluorescent moieties that are highly photostable. In the ideal situation, a fluorescent moiety should emit a stable fluorescence signal, showing no or little deterioration or change of the signal during the course of the experiment. The best fluorescent proteins for live cell imaging can be excited many times, thereby producing a large number of emitted photons before they are destroyed.

“Multi-color brightness and photostability” refers to the brightness and photostability of fluorescent moieties with different emission wavelengths in a multiplexed fluorescence in-situ hybridization system. The fluorescence emission spectral profiles of common fluorescent moieties differ significantly with regard to bandwidth, peak emission wavelength, symmetry, and number of maxima. In multi-color labeled specimens, if the degree of labeling and the intensity of fluorescence emission from the fluorescent moieties is not balanced, brighter signals can overwhelm and penetrate the barrier filters of channels reserved for weaker fluorescent moieties or those with less abundant targets. Thus, it is important to balance the brightness of each fluorescent moiety to ensure a balanced signal. In addition, different fluorescent moieties often have different photostability, and one fluorescent moiety may get photobleached first and render the whole multiplexed fluorescence in-situ hybridization system inoperable. Thus, different fluorescent moieties in a multiplexed fluorescence in-situ hybridization system should have comparable photostability to ensure a stable fluorescence signal. Photostability can be affected by experimental parameters (e.g. excitation light intensity, pH or temperature). A “spot calling algorithm” developed for a quantitative analysis refers to an algorithm that can be used to count the number of particles in an image. In some embodiments, a calibration slide contains a known number of first beads, each first bead having a binding domain for a first probe. In some embodiments, contacting the calibration slide to a saturating amount of first probes creates a calibration slide having one or more fluorescent moieties on each first bead. In some embodiments, the accuracy of the spot calling algorithm can be measured by using the algorithm to count the number of beads on such a calibration slide.

“Conjugation efficiency” between a fluorescent moiety and a probe relates to the stability of the conjugated probe containing the fluorescent moiety. In some embodiments, the fluorescent moiety can be conjugated to the probe using known chemistry, such as via an NHS ester conjugation. The conjugation efficiency can be measured by determining whether the probe has any fluorescent moiety attached, e.g., by measurement of absorbance or fluorescence.

A “balanced signal” refers to when two different fluorescent moieties emit signals of about equal intensity. The fluorescence emission spectral profiles of common fluorescent moieties differ significantly with regard to bandwidth, peak emission wavelength, symmetry, and number of maxima. In multiple labeled specimens, if the degree of labeling and the intensity of fluorescence emission from the fluorescent moieties is not balanced, brighter signals can overwhelm and penetrate the barrier filters of channels reserved for weaker fluorescent moieties or those with less abundant targets. The result is too often a significant contribution from the overstained fluorescent moiety to the image recorded in the channel reserved for a lower intensity probe. To avoid the crosstalk of fluorescence emission, a bead can be designed to have more binding domains for a weaker fluorescent moiety and less binding domains for a stronger fluorescent moiety. In this way, the signals from the weaker and stronger fluorescent moieties are comparable in intensity, generating a balanced signal.

“Brightness” measures the strength of the signal from a certain fluorescent moiety. A fluorescent moiety's brightness is defined by two parameters: its optical absorptivity and quantum yield. The extinction coefficient is a measure of the quantity of absorbed light at a given wavelength. Therefore, a high extinction coefficient will lead to a greater amount of light being absorbed. Quantum yield is the number of emitted photons relative to the number of absorbed photons. Quantum yields of fluorescent molecules commonly employed as probes in microscopy have quantum yields ranging from 0.05 to 1. In general, a high quantum yield is desirable in most imaging applications. The quantum yield of a given fluorescent moiety varies, sometimes to large extremes, with environmental factors, such as metallic ion concentration, pH, and solvent polarity. Fluorescent moieties with high quantum yields, such as rhodamines, display the brightest emissions.

### Methods for Preparing Oligonucleotide-nanoparticle Calibration Slides

In some embodiments, a calibration slide has a substrate and a plurality of beads secured to the surface of the substrate. In some embodiments, the substrate is glass. In some embodiments, the beads are secured to the substrate by chemical cross-linking. In some embodiments, the beads are carboxyl latex beads containing surface carboxyl groups and the substrate is an amine functionalized glass containing surface amines.

In some embodiments, the alignment beads are not auto-fluorescent. In some embodiments, the alignment beads are not auto-fluorescent in about the same wavelength as any readout domain. In some embodiments, alignment beads comprise non-porous silica. In some embodiments, the alignment beads comprise one or more organic polymers. Examples of such organic polymers include, but are not limited to, polystyrene, polyethylene, polypropylene, and poly(vinyl)alcohol. In some embodiments, the alignment comprise a dispersed colloidal suspension of spherical particles comprising amorphous polyisoprene (latex).

In some embodiments, the diameter of the alignment beads is about 0.05 micrometers to about 1 micrometers (μm). The beads can be equal to, or larger than, the pixel size of the image, e.g., 70-120 nm, but need not be larger than about 10 times the pixel size. In some embodiments, the diameter of the beads is about 0.2 μm. In some embodiments, the diameter of the beads is about the same as the diameter of the target spot, thereby having comparable signal intensity to that of the target spot. The signal intensity of the alignment beads can be adjusted to match with the signal intensity of the target region for balanced signal level by adjusting the density of binding sites on the alignment beads. The fluorescence intensity from the labeled beads can be adjusted to match with the fluorescence intensity of the target region for balanced signal level by adjusting the density of probes used to label the beads (i.e., less probes can be used for labeling larger beads). In some embodiments, the density of binding sites on the alignment beads is about 480 to about 2400. In some embodiments, the density of binding sites on the alignment beads is about 1200.

In some embodiments, each targeting domain comprises an oligonucleotide. In some embodiments, each binding site comprises an oligonucleotide. In some embodiments, each analyte comprises an oligonucleotide. In some embodiments, the oligonucleotide is DNA. In some embodiments, the oligonucleotide is RNA. In some embodiments, the each oligonucleotide independently comprises 15 to 30 nucleotide residues.

Attachment of the oligonucleotide binding sites to the alignment beads can be achieved via appropriate chemical coupling reactions which are well known to those skilled in the art. In some embodiments, the chemical functional groups (coupling partners) for a covalent coupling reaction are amine/carboxyl groups in an amide-bond forming reaction. In some embodiments, the coupling partners for a coupling reaction are thiol/maleimide groups in a Michael reaction. In some embodiments, the coupling partners for a coupling reaction are thiol/disulfide groups in a disulfide exchange reaction. In some embodiments, the coupling partners for a coupling reaction are hydroxyl/epoxy groups in an epoxy ring-opening reaction. In some embodiments, the coupling partners for a coupling reaction are amino/epoxy groups in an epoxy ring-opening reaction.

The chemical functional group is linked to the 5′ phosphate group of the oligonucleotides. In some embodiments, the chemical functional group is linked to the 5′ phosphate group of the oligonucleotides with a spacer of about 3 to about 16 carbon atoms, for example, a 3 carbon spacer, a 4 carbon spacer, a 5 carbon spacer, a 6 carbon spacer, a 7 carbon spacer, a 8 carbon spacer, a 9 carbon spacer, a 10 carbon spacer, a 11 carbon spacer, a 12 carbon spacer, a 13 carbon spacer, a 14 carbon spacer, a 15 carbon spacer, or a 16 carbon spacer. In some embodiments, the spacer is a 6 carbon spacer. In some embodiments, the spacer is a 12 carbon spacer.

In some embodiments, the surface charges on the alignment beads can be positive or negative, depending on the identity of the functional groups present. In some embodiments, the alignment beads comprise one or positively charged groups impart positive surface charges to the beads, for example, one or more amidine groups. In some embodiments, the alignment beads comprise one or negatively charged groups impart negative surface charges to the beads, for example, one or more carboxyl and/or sulfate groups. In some embodiments, the alignment beads have no net surface charge, for example, no positively or negatively charged groups, or an equal number of positively and negatively charged groups resulting in no net surface charge.

In some embodiments, the surface charge densities on the alignment beads is about 70 Å per charge group to about 1000 Å per charge group, for example, about 70 Å to about 300 Å per charge group, about 200 Å to about 400 Å per charge group, about 300 Å to about 500 Å per charge group, about 400 Å to about 600 Å per charge group, about 600 Å to about 800 Å per charge group, about 800 Å to about 1,000 Å per charge group, or any value in between.

The chemical coupling reactions are carried out under appropriate reaction conditions (reaction solvent, additives, pH, temperature, reaction time) that favor the progression of the coupling reaction.

In some embodiments, the coupling reactions are carried out in an aqueous buffer. In some embodiments, the aqueous buffer has a pH between about 2 and about 10, for example, about 2, about 3, about 4, about 5, about 6, about 7, about 8, about 9, about 10, or any value in between.

In some embodiments, the aqueous buffer comprises (N-morpholino)ethanesulfonic acid (MES), tris(hydroxymethyl)aminomethane (Tris), or tris(hydroxymethyl)aminomethane-ethylenediaminetetraccetic acid (TE) buffer. In some embodiments, the aqueous buffer is (N-morpholino)ethanesulfonic acid (MES). In some embodiments, the buffer is tris(hydroxymethyl)aminomethane (Tris). In some embodiments, the buffer is tris(hydroxymethyl)aminomethane-ethylenediaminetetraccetic acid (TE) buffer.

In some embodiments, one or more additives are used to promote the coupling reaction. In some embodiments, the one or more additive comprises 1-ethyl-3-(3-dimethylaminopropyl)carbodiimide (EDC), N,N′-diisopropylcarbodiimide (DIC), hydroxybenzotriazole (HOBt), 3-[Bis(dimethylamino)methyliumyl]-3H-benzotriazol-1-oxide hexafluorophosphate (HBTU), (Benzotriazol-1-yloxy)tripyrrolidinophosphonium hexafluorophosphate (PyBOP), bromotripyrrolidinophosphonium hexafluorophosphate (PyBrOP), or a combination of any of the foregoing. In some embodiments, the one or more additives comprises a base, for example, an organic base such as trimethylamine (TEA) or diisopropylethylamine (DIPEA).

In some embodiments, the coupling reactions are carried out at about 0° C. to about 60° C., for example, about 0° C. to about 20° C., about 10° C. to about 30° C., about 20° C. to about 40° C., about 30° C. to about 50° C., about 40° C. to about 60° C., about 0° C., about 5° C., about 10° C., about 15° C., about 20° C., about 25° C. (i.e., “room temperature), about 30° C., about 35° C., about 40° C., about 45° C., about 50° C., about 55° C., about 60° C., or any value in between. In some embodiments, the coupling reactions are carried out at room temperature (about 25° C.). In some embodiments, the coupling reactions are carried out at about 37° C.

Each alignment bead can be modified with one or more different oligonucleotide binding sites using the chemical coupling reactions and conditions described above. The oligonucleotide sequence of each binding site is designed to be the same as the nucleic acid (gene) sequence of interest in the analytes and reverse-complementary to the targeting domains of the probes. Design of the oligonucleotide binding site sequences can be performed using a publicly-available online sequence calculator. In one example the online calculator can be accessed via the internet website: http://reverse-complement.com. For optimal hybridization efficiency, the length of the oligonucleotides is about 15 to 30 residues. oligonucleotides that are too short can cause non-specific binding, whereas and oligonucleotides that are too long require longer hybridization times. In some embodiments, the length of the binding site is 33 oligonucleotide residues. In some embodiments, the length of the binding site is about 15 to 30 oligonucleotide residues.

In some embodiments, the calibration slide can have a plurality of different types of beads. For example, a calibration slide can have a first type of beads having a first binding domain and a second type of beads having a second binding domain. In some embodiments, as a negative control, a calibration slide can also have a third bead having no binding domain. In some embodiments, the calibration slide can have a first type of beads in a first location and a second type of beads in a second location on the calibration slide. In some embodiments, a bead can have a plurality of different types of binding domains. For example, the calibration slide can have (1) a first type of beads having both a first binding domain and a second binding domain; and (2) a second type of beads having both a third binding domain and a fourth binding domain.

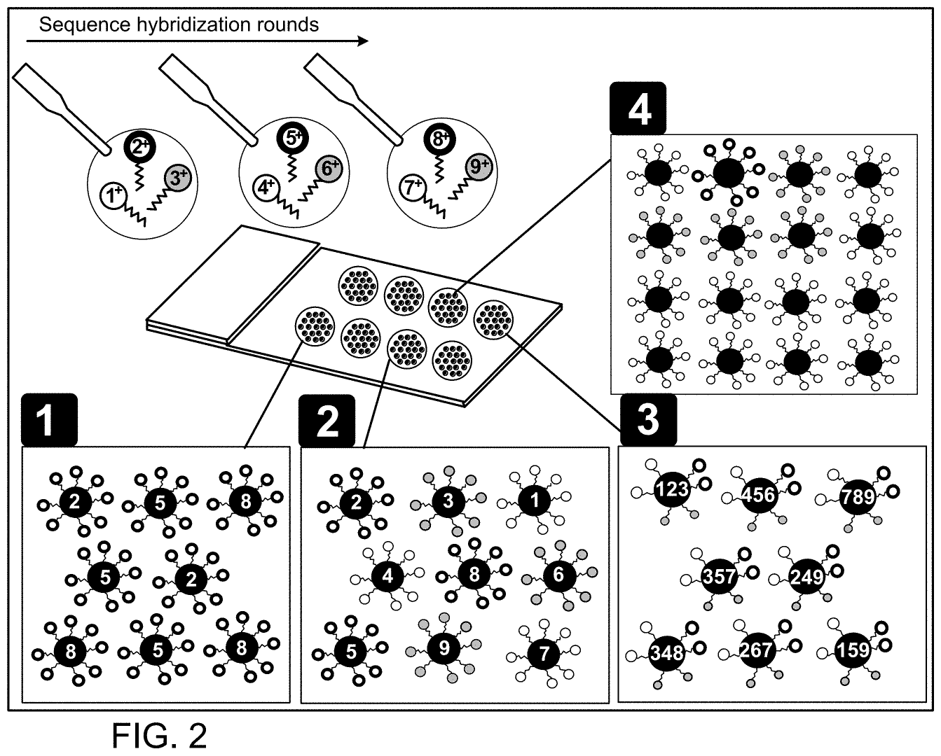

In some embodiments, as shown in FIG. 2, sequential hybridization rounds use different sets of probes that bind to different binding domains. Each set of probes emits the same color, e.g., probe 1 emits a particular color, e.g., red light. Within a particular hybridization round, the different sets of probes emit different colors. For example, in the first hybridization round probe 1 can emit red light, probe 2 can emit green light, and probe 3can emit yellow light. Within separate hybridization rounds, different sets of probes can reuse the same color. For example, probes 1 in the first hybridization round and probe 4 in the second hybridization round can emit the same first color, e.g., red light. Similarly, probe 2 in the first hybridization round and probe 5 in the second hybridization round can emit the same second color that is different from the first color, e.g., green light.

The calibration slide can be separated into different sections containing different types of beads. In some embodiments, a first section of the calibration slide contains beads containing binding domains for probes numbered 2, 5 and 8. These probes 2, 5 and 8 can emit common color of light, e.g., green light. Similarly, a second section of the calibration slide can contain beads containing binding domains for probes numbered 1, 4 and 7. These probes 1, 4 and 7 can emit a common second color of light, e.g., red light, that is different from the first color.

In some embodiments, each bead in the first section only contains one type of binding domain.

In some embodiments, a section of the calibration slide contains beads containing binding domains for probes numbered 1-9.

In some embodiments, a section of the calibration slides contains beads containing binding domains for probes numbered 1-9. In some embodiments, beads in the section contain several types of binding domains.

### Embodiments

In some embodiments, the method of calibrating a fluorescence in-situ hybridization system involves providing a calibration slide having a surface to which a plurality of beads are secured, each bead having a plurality of binding domains configured to specifically bind to a targeting domain of a probe; contacting the calibration slide to a first plurality of first probes, each first probe having a first fluorescent moiety and a first targeting domain such that the first targeting domain binds to a first binding domain of a plurality of first beads; obtaining a first image of the calibration slide; and based on at least the first image and predetermined data concerning the plurality of beads, calibrating the multiplexed fluorescence in-situ hybridization system.

In some embodiments, the method of calibrating a fluorescence in-situ hybridization system further involves contacting the calibration slide to a first plurality of second probes, each second probe having a second fluorescent moiety and a second targeting domain such that the second targeting domain binds to a second binding domain of a plurality of first beads; and obtaining a second image of the calibration slide. In some embodiments, the method of calibrating a fluorescence in-situ hybridization system further involves calibrating the fluorescent in-situ hybridization system based on the first image and the second image. In some embodiments, the method of calibrating a fluorescence in-situ hybridization system further involves contacting the calibration slide to a mixture of first probes and second probes; and obtaining a third image of the calibration slide.

In some embodiments, after contacting the calibration slide to the first probes, the calibration slide is washed by a wash buffer. In some embodiments, the wash buffer can remove unbound first probes. In some embodiments, the first probe has a first targeting domain and a first fluorescent moiety. In some embodiments, after obtaining the first image of the calibration slide containing the first probes, the calibration slide is purged with photo-bleaching. In some embodiments, the first fluorescent moiety becomes unable to fluoresce after the photo-bleaching step.

In some embodiments, the method of calibrating a fluorescence in-situ hybridization system further involves contacting the calibration slide to a second plurality of first probes, each first probe having a first fluorescent moiety and a first targeting domain such that the first targeting domain binds to a first binding domain of a plurality of first beads; and obtaining a fourth image of the calibration slide. In some embodiments, the method of calibrating a fluorescence in-situ hybridization system further involves calibrating the fluorescent in-situ hybridization system based on the first image and the fourth image. In some embodiments, the first plurality of first probes and the second plurality of first probes have different probe concentrations.

In some embodiments, the method of calibrating a fluorescence in-situ hybridization system involves determining the binding efficiency between a probe and a binding domain. In some embodiments, two identical calibration slides are separately contacted to (1) a plurality of first probes with a first targeting domain; and (2) a plurality of second probes with a second targeting domain. In some embodiments, the two identical calibration slides contain beads containing the same amount of first binding domains, which can bind to both the first targeting domain and the second targeting domain. In some embodiments, the first probes and second probes have the same fluorescent moieties. After washing off unbound probes, the two identical calibration slides are separately imaged and compared. Based on the images of the two identical calibration slides, the binding efficiency between the first probe and the first binding domain is characterized.

In some embodiments, the method of calibrating a fluorescence in-situ hybridization system involves determining the conjugation efficiency between a first fluorescent moiety and a first probe. In some embodiments, two identical calibration slides are separately contacted to (1) a plurality of first probes with a first fluorescent moiety; and (2) a plurality of second probes with a second fluorescent moiety. In some embodiments, the first probes and the second probes have the same targeting domain. After washing off unbound probes, the two identical calibration slides are separately imaged and compared. Based on the images of the two identical calibration slides, the conjugation efficiency between a first fluorescent moiety and a first probe is determined.

In some embodiments, the method of calibrating a fluorescence in-situ hybridization system involves determining the appropriate image acquisition conditions. In some embodiments, two identical calibration slides are separately contacted to a plurality of first probes. After washing off unbound probes, the two identical calibration slides are separately imaged under different image acquisition conditions. Based on the images of the two identical calibration slides, the appropriate image acquisition condition is characterized.

In some embodiments, the method of calibrating a fluorescence in-situ hybridization system involves determining multi-color brightness and photostability of the fluorescent moieties. In some embodiments, two identical calibration slides are separately contacted to a mixture of first probes and second probes. In some embodiments, the first probe has a first fluorescent moiety and a first targeting domain that can specifically bind to a first binding domain on a bead on the calibration slides. In some embodiments, the second probe has a second fluorescent moiety and a second targeting domain that can specifically bind to a second binding domain on a bead on the calibration slides. In some embodiments, the two slides are imaged at either different time points or under different imaging conditions. In some embodiments, the images are compared in order to determine multi-color brightness and photostability of the first and second fluorescent moieties.

In some embodiments, the method of calibrating a fluorescence in-situ hybridization system involves determining the optimal number of binding domains on a bead to generate particles of various signal levels and to reach a balanced signal. In some embodiments, a first calibration slide and a second calibration slide are separately contacted to a mixture of first probes and second probes. In some embodiments, the first calibration slide has a plurality of first beads, each first bead containing a first number of first binding domains, where each first binding domain can specifically bind to a first targeting domain on a first probe. In some embodiments, the second calibration slide has a plurality of second beads, each second bead containing a second number of second binding domains, where each second binding domain can specifically bind to a second targeting domain on a second probe. In some embodiments, the first and second calibration slides are imaged under the same image acquisition conditions. In some embodiments, the images are analyzed to determine the optimal number of binding domains on a bead in order to reach a balanced signal.

In some embodiments, the method of calibrating a fluorescence in-situ hybridization system involves generating template images to test spot calling algorithm developed for a quantitative analysis. In some embodiments, a first calibration slide and a second calibration slide are separately contacted to a mixture of first probes and second probes. In some embodiments, the first calibration slide has a plurality of first beads containing a first number of first binding domains, where each first binding domain can specifically bind to a first targeting domain on a first probe. In some embodiments, the second calibration slide has a plurality of second beads containing a second number of second binding domains, where each second binding domain can specifically bind to a second targeting domain on a second probe. In some embodiments, the number of first beads and second beads are known. In some embodiments, the number of first beads on the first calibration slide is different from the number of second beads on the second calibration slide. In some embodiments, the first and second calibration slides are imaged under the same image acquisition conditions. In some embodiments, the images are analyzed to test spot calling algorithm developed for a quantitative analysis.

In some embodiments, as shown in FIG. 2, the method of calibrating a fluorescence in-situ hybridization system involves sequential hybridization rounds. In some embodiments, as shown in FIG. 2, the calibration slide is separated into different sections containing different types of beads. In some embodiments, a first section of the calibration slide contains beads containing binding domains for probes numbered 2, 5 and 8. In some embodiments, each bead in the first section only contains one type of binding domain. In some embodiments, a second section of the calibration slide contains beads containing binding domains for probes numbered 1-9. In some embodiments, each bead in the second section only contains one type of binding domain. In some embodiments, a third section of the calibration slides contains beads containing binding domains for probes numbered 1-9. In some embodiments, one bead in the third section contains several types of different binding domains. In some embodiments, the calibration slide is contacted sequentially to a mixture of probes numbered 1-3, a mixture of probes numbered 4-6 and a mixture of probes numbered 7-9. In some embodiments, unbound probes are washed off before the calibration slide is contacted to a new mixture of probes. In some embodiments, the first section of the calibration slide retains probes numbered 2, 5 and 8. In some embodiments, the second section and third section of the slides retain probes numbered 1-9.

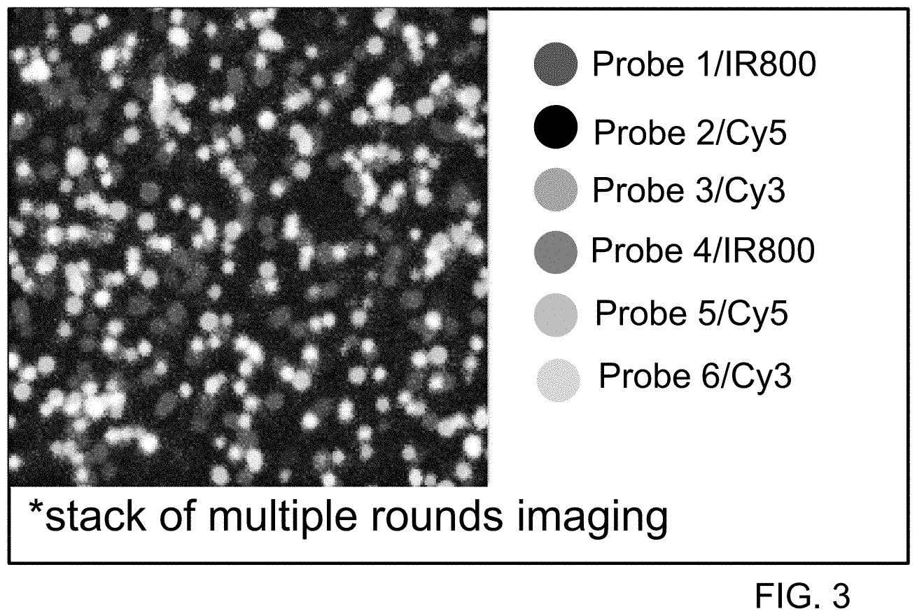

In some embodiments, as shown in FIG. 3, the method of calibrating a fluorescence in-situ hybridization system involves multiple rounds of imaging with different color channels. In some embodiments, the calibration slide contains binding domains for six different types of probes. In some embodiments, the six different types of probes contain six different fluorescent groups with different excitation and emission wavelengths. In some embodiments, six different images are taken with six different color channels. In some embodiments, as shown in FIG. 3, the six different images from six different color channels are overplayed to create a stack of multiple rounds of imaging.

### Automated Microfluidics and Image Acquisition System.

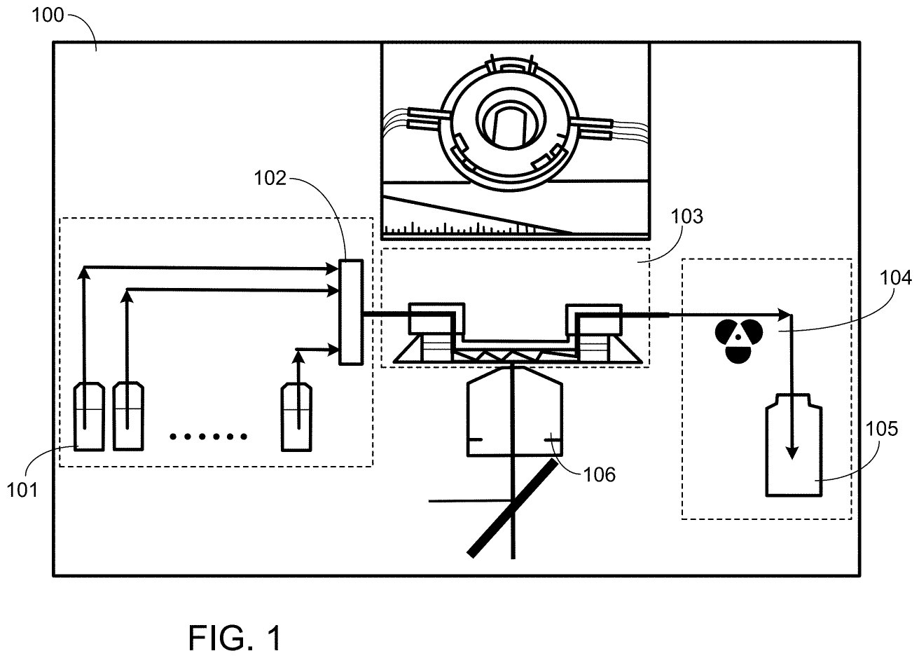

In some embodiments, as shown in FIG. 1, an automated microfluidics and image acquisition system 100 is used to implement the method of calibrating a fluorescence in-situ hybridization system. In some embodiments, the calibration slide is placed in the flowcell sample chamber 103, which is under the observation of a microscope objective 106 of a microscope. In some embodiments, the microscope can be epifluorescence, laser scanning confocal, or spinning disk confocal microscopes. In some embodiments, the automated microfluidics system 100 enables computer controlled delivery of various fluids onto the calibration slide in the flowcell sample chamber 103. In some embodiments, a valve controlled delivery system 102 can deliver solutions containing various probes containing fluorescent moieties from fluid reservoir 101 onto the calibration slide in the flowcell sample chamber 103. In some embodiments, a valve controlled delivery system 102 can deliver different buffers (e.g., wash buffer, imaging buffer, bleach buffer) onto the calibration slide in the flowcell sample chamber 103. In some embodiments, valve positioners can be employed to control which fluid is delivered onto the calibration slide in the flowcell sample chamber 103. In some embodiments, a peristaltic pump 104 is used to pump unwanted fluids out of the flowcell sample chamber 103. In some embodiments, unwanted fluids are collected in a waste bottle 105.

### Example. Functionalization of 0.2-μm Carboxyl Latex Beads with 5′-amino-oligonucleotides

A 5′-amino oligonucleotide, wherein the amino group is covalently linked to the 5′-phosphate group of the oligonucleotide with a 12-carbon spacer in between, can be procured from commercial suppliers. The oligonucleotides are prepared synthetically via conventional automated solid-state oligonucleotide synthesis, and the desired sequences can be specified as part of the oligonucleotide synthesis. The oligonucleotides can be supplied as dry powders, or as aqueous solutions in water or other aqueous buffers. The oligonucleotides as dry powders are preferred as they are more stable than when stored as an aqueous solution.

Oligonucleotides as dry powders were re-suspended in ultra-pure water to a final concentration of 1 mM, aliquoted into single-use vials and stored at −20° C. Frozen aliquots were thawed prior to use and were not re-frozen to avoid multiple freeze-thaw cycles. Upon thawing, 2.5-μL of the 1 mM (corresponding to 2.5 nmol) of each oligonucleotide solution was transferred to a 1.8-mL conical tube via an automatic micropipette. Multiple different oligonucleotide solutions can be combined in a single conical tube for each run of alignment bead functionalization reaction. To the combined oligonucleotides solution was added 1 volume of MES buffer; for example, if 16 oligonucleotide solutions were combined, 40-μL (15*2.5-μL) of the MES buffer would be added. The resulting solution was vortexed gently to mix the components.

1 pmol of a 4% (w/v) aqueous suspension of the 0.2-μm diameter carboxyl latex beads (Catalog no. C37486, Molecular Probes) was transferred to a 1.8-mL conical tube via a micropipette, to which 3 volumes of MES buffer was added, resulting in a 1% (w/v) bead suspension. The bead suspension was centrifuged at 12,000 g for 15 min to fully precipitate the beads. The supernatant was discarded, and the pellet was re-suspended in 400-μL of MES buffer with gentle vortexing. The resulting bead suspension was added to the oligonucleotide/MES solution prepared as described above.

A stock solution of 10 mg/mL EDC in ultrapure water was prepared and 5-μL (corresponding to about 250 nmol of EDC) of the stock solution was added to the bead/oligonucleotide mixture as described above to initiate the coupling reaction. The resulting mixture vortexed at 200-1000 rpm at room temperature for 15min, incubated at 4° C. for 3 hours, and then terminated by adding 50-μL of 1M Tris buffer to the reaction mixture followed by vortexing at 200-1000 rpm for 15 min. The mixture was then centrifuged at 12,000 g for 15 min, upon which the supernatant was discarded and the pellet re-suspended in 500-μL of TE buffer. The centrifugation/re-suspension steps were repeated once and the resulting oligonucleotide-functionalized beads were stored at refrigerated temperature (2-8° C.) until used.

### FISH Imaging System

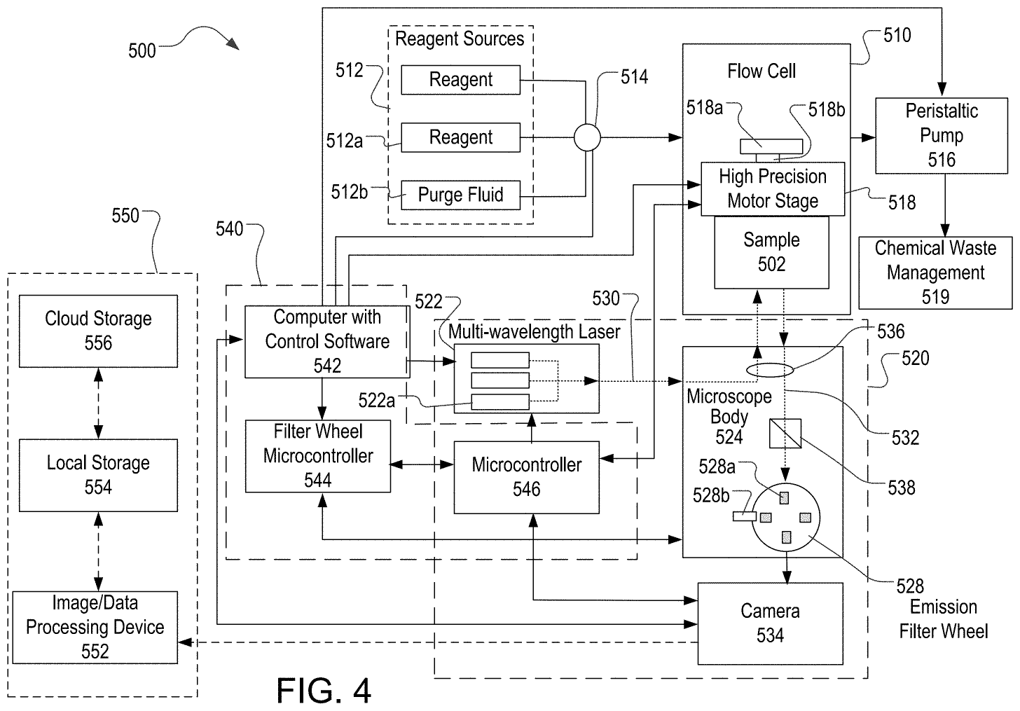

FIG. 4 illustrates a multiplexed fluorescent in-situ hybridization (mFISH) imaging and image processing apparatus 500.The mFISH imaging and image processing apparatus 500 can correspond to the microfluidics and image acquisition system 100. The mFISH imaging and image processing apparatus 500 includes a flow cell 510 to hold a sample 502, a fluorescence microscope 520 to obtain images of the sample 502, and a control system 540 to control operation of the various components of the mFISH imaging and image processing apparatus 500. The control system 540 can include a computer 542, e.g., having a memory, processor, etc., that executes control software.

The fluorescence microscope 520 includes an excitation light source 522 that can generate excitation light 530 of multiple different wavelengths. In particular, the excitation light source 522 can generate narrow-bandwidth light beams having different wavelengths at different times. For example, the excitation light source 522 can be provided by a multi-wavelength continuous wave laser system, e.g., multiple laser modules 522a that can be independently activated to generate laser beams of different wavelengths. Output from the laser modules 522a can be multiplexed into a common light beam path.

The fluorescence microscope 520 includes a microscope body 524 that includes the various optical components to direct the excitation light from the light source 522 to the flow cell 510. For example, excitation light from the light source 522 can be coupled into a multimode fiber, refocused and expanded by a set of lenses, then directed into the sample 502 by a core imaging component, such as a high numerical aperture (NA) objective lens 536. When the excitation channel needs to be switched, one of the multiple laser modules 522a can be deactivated and another laser module 522a can be activated, with synchronization among the devices accomplished by one or more microcontrollers 544, 546.

The objective lens 536, or the entire microscope body 524, can be installed on vertically movable mount coupled to a Z-drive actuator. Adjustment of the Z-position, e.g., by a microcontroller 546 controlling the Z-drive actuator, can enable fine tuning of focal position. Alternatively, or in addition, the flow cell 510 (or a stage 518 supporting the sample in the flow cell 510) could be vertically movable by a Z-drive actuator 518b, e.g., an axial piezo stage. Such a piezo stage can permit precise and swift multi-plane image acquisition.

The sample 502 to be imaged is positioned in the flow cell 510. The flow cell 510 can be a chamber with cross-sectional area (parallel to the object or image plane of the microscope) with and area of about 2 cm by 2 cm. The sample 502 can be supported on a stage 518 within the flow cell, and the stage (or the entire flow cell) can be laterally movable, e.g., by a pair of linear actuators 518a to permit XY motion. This permits acquisition of images of the sample 502 in different laterally offset fields of view (FOVs). Alternatively, the microscope body 524 could be carried on a laterally movable stage.

An entrance to the flow cell 510 is connected to a set of hybridization reagents sources 512. A multi-valve positioner 514 can be controlled by the controller 540 to switch between sources to select which reagent 512 a is supplied to the flow cell 510. Each reagent includes a different set of one or more oligonucleotide probes, e.g., readout probes. Each probe targets a different RNA sequence of interest, and has a different set of one or more fluorescent materials, e.g., phosphors, that are excited by different combinations of wavelengths. In addition to the reagents 512a, there can be a source of a purge fluid 512 b, e.g., deionized (“DI”) water.

An exit to the flow cell 510 is connected to a pump 516, e.g., a peristaltic pump, which is also controlled by the controller 540 to control flow of liquid, e.g., the reagent or purge fluid, through the flow cell 510. Used solution from the flow cell 510 can be passed by the pump 516 to a chemical waste management subsystem 519.

In operation, the controller 540 causes the light source 522 to emit the excitation light 530, which causes fluorescence of fluorescent material in the sample 502, e.g., fluorescence of the probes that are bound to RNA in the sample and that are excited by the wavelength of the excitation light. The emitted fluorescent light 532, as well as back propagating excitation light, e.g., excitation light scattered from the sample, stage, etc., is collected by an objective lens 536 of the microscope body 524.

The collected light can be filtered by a multi-band dichroic mirror 538 in the microscope body 524 to separate the emitted fluorescent light from the back propagating illumination light, and the emitted fluorescent light is passed to a camera 534. The multi-band dichroic mirror 538 can include a pass band for each emission wavelength expected from the probes, e.g., the readout probes, under the variety of excitation wavelengths. Use of a single multi-band dichroic mirror (as compared to multiple dichroic mirrors or a movable dichroic mirror) can provide improved system stability.

The camera 534 can be a high resolution (e.g., 2048×2048 pixel) CMOS (e.g., a scientific CMOS) camera, and can be installed at the immediate image plane of the objective. Other camera types, e.g., CCD, may be possible. When triggered by a signal, e.g., from a microcontroller, image data from the camera can be captured, e.g., sent to an image processing system 550. Thus, the camera 534 can collect a sequence of images from the sample.

To further remove residual excitation light and minimize cross talk between excitation channels, each laser emission wavelength can be paired with a corresponding band-pass emission filter 528a. Each filter 528a can have a wavelength of 10-50 nm, e.g., 14-32 nm. In some implementations, a filter is narrower than the bandwidth of the fluorescent material of the probe resulting from the excitation, e.g., if the fluorescent material of the probe has a long trailing spectral profile.

The filters are installed on a high-speed filter wheel 528 that is rotatable by an actuator. The filter wheel 528 can be installed at the infinity space to minimize optical aberration in the imaging path. After passing the emission filter of the filter wheel 528, the cleaned fluorescence signals can be refocused by a tube lens and captured by the camera 534. The dichroic mirror 538 can be positioned in the light path between the objective lens 538 and the filter wheel 528.

To facilitate high speed, synchronized operation of the system, the control system 540 can include two microcontrollers 544, 546 that are employed to send trigger signals, e.g., TTL signals, to the components of the fluorescence microscope 520 in a coordinated manner. The first microcontroller 544 is directly run by the computer 542, and triggers actuator 528b of the filter wheel 528 to switch emission filters 528a at different color channels. The first microcontroller 544 or the computer 542 can trigger the second microcontroller 546, which sends digital signals to the light source 522 in order to control which wavelength of light is passed to the sample 502. For example, the second microcontroller 546 can send on/off signals to the individual laser modules of the light source 522 to control which laser module is active, and thus control which wavelength of light is used for the excitation light. After completion of switching to a new excitation channel, the second microcontroller 546 controls the motor for the piezo stage 518b to select the imaging height. Finally the second microcontroller 546 sends a trigger signal to the camera 534 for image acquisition.

Communication between the computer 542 and the device components of the mFISH apparatus 500 is coordinated by the control software. This control software can integrate drivers of all the device components into a single framework, and thus can allow a user to operate the imaging system as a single instrument (instead of having to separately control many devices).

The control software supports interactive operations of the microscope and instant visualization of imaging results. In addition, the control software can provide a programming interface which allows users to design and automate their imaging workflow. A set of default workflow scripts can be designated in the scripting language.

In some implementations, the control system 540 is configured, i.e., by the control software and/or the workflow script, to acquire fluorescence images (also termed simply “collected images” or simply “images”) in loops in the following order (from innermost loop to outermost loop): z-axis, color channel, lateral position, and reagent.

These loops may be represented by the pseudocode in Table 1, below.

TABLE 1
example control system loop pseudocode

```
for h = 1:N_hybridization
  % multiple hybridizations
  for f = 1:N_FOVs
    % multiple lateral field-of-views
    for c = 1:N_channels
      % multiple color channels
      for z = 1:N_planes
        % multiple z planes
        Acquire image(h, f, c, z);
      end % end for z
    end % end for c
  end % end for f
end % end for h
```

For the z-axis loop, the control system 540 causes the stage 518 to step through multiple vertical positions. Because the vertical position of the stage 518 is controlled by a piezoelectric actuator, the time required to adjust positions is small and each step in this loop can be extremely fast.

First, the sample can be sufficiently thick, e.g., a few microns, that multiple image planes through the sample may be desirable. For example, multiple layers of cells can be present, or even within a cell there may be a vertical variation in gene expression. Moreover, for thin samples, the vertical position of the focal plane may not be known in advance, e.g., due to thermal drift. In addition, the sample 502 may vertically drift within the flow cell 510. Imaging at multiple Z-axis positions can ensure most of the cells in a thick sample are covered, and can help identify the best focal position in a thin sample.

For the color channel loop, the control system 540 causes the light source 522 to step through different wavelengths of excitation light. For example, one of the laser modules is activated, the other laser modules are deactivated, and the emission filter wheel 528 is rotated to bring the appropriate filter into the optical path of the light between the sample 502 and the camera 534.

For the lateral position, the control system 540 causes the light source 522 to step through different lateral positions in order to obtain different fields of view (FOVs) of the sample. For example, at each step of the loop, the linear actuators supporting the stage 518 can be driven to shift the stage laterally. In some implementations, the control system 540 number of steps and lateral motion is selected such that the accumulated FOVs to cover the entire sample 502. In some implementations, the lateral motion is selected such that FOVs partially overlap.

For the reagent, the control system 540 causes the mFISH apparatus 500 to step through multiple different available reagents. For example, at each step of the loop, the control system 540 can control the valve 514 to connect the flow cell 510 to the purge fluid 512b, cause the pump 516 to draw the purge fluid through the cell for a first period of time to purge the current reagent, then control the valve 514 to connect the flow cell 510 to different new reagent, and then draw the new reagent through the cell for a second period of time sufficient for the probes in the new reagent to bind to the appropriate RNA sequences. Because some time is required to purge the flow cell and for the probes in the new reagent to bind, the time required to adjust reagents can be longer than the time required for other steps in the process, e.g., as compared to adjusting the lateral position, color channel or z-axis.

As a result, a fluorescence image is acquired for each combination of possible values for the z-axis, color channel (excitation wavelength), lateral FOV, and reagent. Because the innermost loop has the fastest adjustment time, and the successively surrounding loops are of successively slower adjustment time, this configuration can provide the most time efficient technique to acquire the images for the combination of values for these parameters.

A data processing system 550 is used to process the images and determine gene expression to generate the spatial transcriptomic data. At a minimum, the data processing system 550 includes a data processing device 552, e.g., one or more processors controlled by software stored on a computer readable medium, and a local storage device 554, e.g., non-volatile computer readable media, that receives the images acquired by the camera 534. For example, the data processing device 552 can be a work station with GPU processors or FPGA boards installed. The data processing system 550 can also be connected through a network to remote storage 556, e.g., through the Internet to cloud storage.

The data processing system 550 can process the images as described in more detail below. For instance, the data processing system 550 can perform one or more steps to stich images from different FOVs together.

In some implementations, the data processing system 550 performs on-the-fly image processing as the images are received. In particular, while data acquisition is in progress, the data processing device 552 can perform image pre-processing steps, such as filtering and deconvolution, that can be performed on the image data in the storage device 554 but which do not require the entire data set. Because filtering and deconvolution can be a major bottleneck in the data processing pipeline, pre-processing as image acquisition is occurring can significantly shorten the offline processing time and thus improve the throughput.

### Calibration Process

In general, calibration can be performed by taking two calibration slides that have identically prepared beads, taking images of the slides under different conditions, and comparing the images. In particular, the intensity values of the light emitted from portions of the two images corresponding to the beads can be measured. A ratio of intensity values can then be calculated and stored for use in calibration. For example, in a later mFISH operation for imaging of a biological sample using the second operating conditions, intensity values from portions of the image corresponding to the sample can be multiplied by the ratio to convert the intensity values to a normalized values. The normalized values can then be used for calculation of aspects of the biological sample, e.g., degree of expression of the targeted nucleotide sequence in the sample, or presences of an expressed gene in the sample.

This technique can be used to calibrate between probes that target the same target oligionucleotide sequence, but may have different binding efficiencies. Two calibration slides are prepared with identical beads that include binding domains with the same target oligionucleotide sequence. In particular the beads of the two calibration slides have the same density and/or amount of first binding domains. The first slide is exposed to a plurality of first probes with a first oligionucleotide sequence that can bind to the target oligionucleotide sequence, and the second slide is exposed to a plurality of second probes with a different second oligionucleotide sequence that can also bind to the target oligionucleotide sequence. The first probes and second probes can use the same fluorophore. After washing off unbound probes, the two calibration slides are separately imaged to provide first and second images. Portions of the first image corresponding to the beads provide a first intensity value, and portions of the second image corresponding to the beads provide a second intensity value. The ratio of first and second intensity values thus provides a measure of the relative binding efficiency of the first and second probes, and can be stored for use in calibration during FISH imaging of biological samples using probes that have the first or second oligionucleotide sequence.

This technique can be used to calibrate between probes that target the same target oligionucleotide sequence, but may have different conjugation efficiencies. Two calibration slides are prepared with identical beads that include binding domains with the same target oligionucleotide sequence. In particular the beads of the two calibration slides have the same density and/or amount of first binding domains. The first slide is exposed to a plurality of first probes with an oligionucleotide sequence that can bind to the target oligionucleotide sequence and a first fluorophore, and the second slide is exposed to a plurality of second probes with the same oligionucleotide sequence but with a different second fluorophore. After washing off unbound probes, the two calibration slides are separately imaged to provide first and second images. Portions of the first image corresponding to the beads provide a first intensity value, and portions of the second image corresponding to the beads provide a second intensity value. The ratio of first and second intensity values thus provides a measure of the relative conjugation efficiency of the first and second probes, and can be stored for use in calibration during FISH imaging of biological samples using the probes that have the first or second fluorophore.

This technique can be used to calibrate between different image acquisition conditions. Again, two calibration slides are prepared with identical beads that include binding domains with the same target oligonucleotide sequence and the same density and/or amount of first binding domains. The first slide and second slide are exposed to identical probes, but under different operating conditions, e.g., different excitation wavelengths, different excitation intensities, different chemistries in the flow cell, different flow rate, different concentration of probes, different incubation times, or different temperatures. It may be useful to vary only one parameter of the imaging acquisition conditions between the two slides, e.g., just the excitation wavelength, or just the excitation intensity, while other parameters remain the same. After washing off unbound probes, the two calibration slides are separately imaged to provide first and second images. Portions of the first image corresponding to the beads provide a first intensity value, and portions of the second image corresponding to the beads provide a second intensity value. The ratio of first and second intensity values thus provides a measure of the effect of the change in operating conditions, and can be stored for use in calibration during FISH imaging of biological samples using the various operating conditions.

This technique can be used to calibrate between fluorophores having different photostability. A calibration slides is prepared with identical beads that include both first binding domains with a first same target oligionucleotide sequence and second binding domains with a second target oligionucleotide sequence. The calibration slide is exposed to a mixture of first probes and second probes. The first probes have a first fluorophore and a first oligionucleotide sequence that can bind to the first target oligionucleotide sequence, and the second probes have a different second fluorophore and a different second oligionucleotide sequence that can bind to the second target oligionucleotide sequence. The first fluorophore and the second fluorophore can emit light at different wavelengths. After washing off unbound probes, the calibration slide is imaged at a first time to provide a first image, and then later imaged at second time to provide a second image. Portions of the first image corresponding to the beads provide a first intensity value, and portions of the second image corresponding to the beads provide a second intensity value. The ratio of first and second intensity values thus provides a measure of the relative photostability of the first and second fluorophores, and can be stored for use in calibration during FISH imaging of biological samples using probes that have the first and second fluorophores.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous

What is claimed is:

1. A calibration slide for calibrating a multiplexed fluorescence in-situ hybridization (FISH) system, the calibration slide comprising:
a substrate having a surface; and
a first plurality of beads on the surface, wherein each bead of the first plurality of beads has at least one binding domain to be bound to a corresponding probe that has a tag and a targeting domain that binds to a binding domain.

2. The calibration slide of claim 1, wherein each bead of the first plurality of beads has exactly one binding domain.

3. The calibration slide of claim 2, wherein the first plurality of beads include first beads having a common first binding domain and second beads having a common second binding domain, the first and second beads having different binding domains.

4. The calibration slide of claim 3, wherein the first beads and the second beads are intermingled in an area on the surface.

5. The calibration slide of claim 3, wherein the first binding domain is configured to bind to a first targeting domain of a first probe that has a first tag that includes a photoluminescent moiety that emits light of a color and the second binding domain is configured to bind to a second targeting domain of a second probe that has a second tag that includes a photoluminescent moiety that emits light of the same color.

6. The calibration slide of claim 3, further comprising a second plurality of beads on the surface, wherein each bead of the second plurality of beads has at least one binding domain to be bound to a corresponding probe that has a tag and a targeting domain that binds to a binding domain.

7. The calibration slide of claim 6, wherein each bead of the second plurality of beads has exactly one binding domain.

8. The calibration slide of claim 7, wherein the second plurality of beads include third beads having a common third binding domain and fourth beads having a common fourth binding domain, the third and fourth beads having different binding domains.

9. The calibration slide of claim 8, wherein the third binding domain is configured to bind to a third targeting domain of a first probe that has a third tag that includes a photoluminescent moiety that emits light of a second color and the fourth binding domain is configured to bind to a fourth targeting domain of a fourth probe that has a fourth tag that includes a photoluminescent moiety that emits light of the same second color, wherein the first color is different from the second color.

10. The calibration slide of claim 9, wherein the first plurality of beads are located in a first portion of the surface the first plurality of beads are located in a second non-overlapping portion of the surface.

11. The calibration slide of claim 9, wherein the first plurality of beads and the second plurality of beads are intermingled in an area on the surface.

12. The calibration slide of claim 1, wherein each bead of the first plurality of beads has exactly a first binding domain and a second binding domain.

13. The calibration slide of claim 3, wherein the first binding domain is configured to bind to a first targeting domain of a first probe that has a first tag that includes a photoluminescent moiety that emits light of a first color and the second binding domain is configured to bind to a second targeting domain of a second probe that has a second tag that includes a photoluminescent moiety that emits light of a different second color.

14. The calibration slide of claim 1, wherein the surface of the calibration slide has a third plurality of beads that have no binding domains.

15. The calibration slide of claim 1, wherein each binding domain independently comprises a nucleic acid.

16. The calibration slide of claim 1, wherein each binding domain independently comprises an oligonucleotide.

17. The calibration slide of claim 1, wherein each binding domain independently comprises an oligonucleotide.

18. The calibration slide of claim 17, wherein each oligonucleotide is independently DNA or RNA.

19. The calibration slide of claim 17, wherein each oligonucleotide independently comprises 15-30 residues.

20. The calibration slide of claim 1, wherein a bead of the plurality of beads comprises a carboxyl latex core.

21. The calibration slide of claim 1, wherein a bead of the plurality of beads has a diameter of between 0.05 μm and 1 μm.
